(19) United States
(12) Patent Application Publication
(10) Pub. No.: US 2024/0342711 A1
(43) Pub. Date: Oct. 17, 2024

(54) SMALL VOLUME FLUIDIC DEVICES

(71) Applicant: 3M INNOVATIVE PROPERTIES COMPANY, St. Paul, MN (US)

(72) Inventors: Steven P. Swanson, Blaine, MN (US); Kurt J. Halverson, Lake Elmo, MN (US); Caleb T. Nelson, McKinney, TX (US)

(21) Appl. No.: 18/292,397

(22) PCT Filed: Aug. 15, 2022
(86) PCT No.: PCT/IB2022/057635

§ 371 Date: Jan. 26, 2024

Related U.S. Application Data
(60) Provisional application No. 63/250,377, filed on Sep. 30, 2021.

(51) Int. Cl. B01L 3/00; B32B 7/14; B32B 27/08; B32B 27/18; B32B 27/30; B32B 27/32; B32B 27/36; B32B 27/40

(52) U.S. Cl. CPC B01L 3/50273; B32B 7/14; B32B 27/08; B32B 27/18; B32B 27/308; B32B 27/32; B32B 27/36; B32B 27/40; B01L 2300/161; B01L 2400/0406; B32B 2255/10; B32B 2255/205; B32B 2270/00; B32B 2307/538; B32B 2307/728; B32B 2535/00

(57) ABSTRACT

The present disclosure provides fluidic devices. A fluidic device includes a) a first bondable polymeric layer having a first major surface that is substantially planar; b) a second polymeric layer having a first major surface that is substantially planar; and c) a hydrophilic mask material disposed on a first portion of the first major surface of the first bondable polymeric layer. A surface of the hydrophilic mask material exhibits an advancing contact angle with water of less than 90 degrees. A second portion of the first major surface of the first bondable polymeric layer is bonded to a first portion of the first major surface of the second polymeric layer. The hydrophilic mask material and a second portion of the first major surface of the second polymeric layer are in direct contact with each other at at least one point. An open volume is defined by interstitial space located between the hydrophilic mask material and the second portion of the first major surface of the second polymeric layer. The open volume comprises two or more openings and at least one of the openings is located at an edge of the first bondable polymeric layer. The fluidic devices can be formed to have small volumes for use as precision fluidic devices, such as blood glucose testing strips.

### TECHNICAL FIELD

The present disclosure broadly relates to fluidic devices for use with small fluid volumes.

### BACKGROUND

Currently die cutting and rotary converting processes are often utilized for manufacturing of fluidic devices used in diagnostic and wearable devices. These processes are limited in their ability to produce small and complex structures and often require multiple individual film constructions that are costly and difficult to assemble. For instance, a common approach to forming a fluidic device chamber is cutting a small notch out of a double coated tape, laminating the tape to a bottom layer with the notch aligned over a sensor, followed by laminating a hydrophilic cover film to form the top of the chamber. However, there is a practical limit to die cutting small features from tapes, making it challenging to reduce the volume of sample required to less than approximately 1 microliter. The volume of the chamber is defined by the thickness of the double coated tape, requiring precise caliper control that is difficult to achieve with adhesive coating processes. Further, thin layers of adhesives tend to have lower bond strength than thicker layers. Using a thin or ultra thin film also risks stretching or breaking of the film during handling in manufacturing of the fluidic device. In addition to the challenges associated with fluidic device assembly, there are also materials issues associated with utilizing double coated tapes to form capillary features. It is challenging, for instance, to render the sidewalls formed by die cutting a tape hydrophilic, therefore the surface energy of the cover film must be very high to induce spontaneous capillary action of aqueous samples such as body fluids.

### SUMMARY

In a first aspect, the present disclosure provides a fluidic device. The fluidic device includes a) a first bondable polymeric layer having a first major surface that is substantially planar; b) a second polymeric layer having a first major surface that is substantially planar; and c) a hydrophilic mask material disposed on a first portion of the first major surface of the first bondable polymeric layer. A surface of the hydrophilic mask material exhibits an advancing contact angle with water of less than 90 degrees. A second portion of the first major surface of the first bondable polymeric layer is bonded to a first portion of the first major surface of the second polymeric layer. The hydrophilic mask material and a second portion of the first major surface of the second polymeric layer are in direct contact with each other at at least one point. An open volume is defined by interstitial space located between the hydrophilic mask material and the second portion of the first major surface of the second polymeric layer. The open volume includes two or more openings and at least one of the openings is located at an edge of the first bondable polymeric layer.

It has been discovered that it is possible to prepare fluidic devices for use with small volumes of fluid by bonding select portions of two (e.g., substantially) planar layers and utilizing the interstitial space between one or more unbonded (e.g., hydrophilic) portions for fluid flow. This provides at least one advantageous property of increased performance by using precise, lower volumes of sample than could be achieved by current fabrication methods, as well as not requiring the formation of a cavity in the device. The fluidic devices may be formed by a simple process, which reduces the number of input materials required, for instance as compared to die cutting and rotary converting processes.

Fluidic devices according to the present disclosure do not require a cavity, thus do not require (e.g., vertical) side walls, eliminating a complexity of geometry as compared to articles or devices including a cavity having side walls. Moreover, these fluidic devices allow for transport of liquid volumes much smaller than current commercialized designs, namely through interstitial transport between two (substantially) flat surfaces.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1A is an exploded generalized schematic diagram of an exemplary fluidic device.

FIG. 1B is a generalized schematic diagram of a cross-section of a portion of the exemplary fluidic device of FIG. 1A.

FIG. 1C is a generalized schematic diagram of another cross-section of a portion of the exemplary fluidic device of FIG. 1A.

FIG. 2A is a stereo microscope image of a portion of an exemplary fluidic device prior to contact with a fluid.

FIG. 2B is a stereo microscope image of a portion of the exemplary fluidic device of FIG. 2A after 0.5 seconds of contact with a fluid.

FIG. 2C is a stereo microscope image of a portion of the exemplary fluidic device of FIG. 2A after 1 second of contact with a fluid.

FIG. 3 is a generalized schematic diagram of a second polymeric layer including a detector having an electrode.

FIG. 4A is a photograph of tapes comprising three different patterned masks according to the present disclosure.

FIG. 4B is a photograph of the sample of Example 1 prior to cutting the exemplary fluidic device from the remainder of the sample, according to the present disclosure.

FIG. 4C is a stereo microscope image of a portion of the fluidic device of Example 1 prior to contact with a fluid.

FIG. 4D is a stereo microscope image of a portion of the fluidic device of Example 1 after 1.685 seconds of contact with a fluid.

FIG. 4E is a stereo microscope image of a portion of the fluidic device of Example 1 after 3.370 seconds of contact with a fluid.

FIG. 5A is a photograph of the sample of Example 2 prior to cutting the exemplary fluidic device from the remainder of the sample, according to the present disclosure.

FIG. 5B is a stereo microscope image of a portion of the fluidic device of Example 2 after 0.062 seconds of contact with a fluid.

FIG. 5C is a stereo microscope image of a portion of the fluidic device of Example 2 after 3.577 seconds of contact with a fluid.

FIG. 5D is a stereo microscope image of a portion of the fluidic device of Example 2 after 4.555 seconds of contact with a fluid.

FIG. 5E is a stereo microscope image of a portion of the fluidic device of Example 2 after 7.051 seconds of contact with a fluid.

FIG. 6A is a photograph of the sample of Example 3 prior to cutting the exemplary fluidic device from the remainder of the sample, according to the present disclosure.

FIG. 6B is a stereo microscope image of a portion of the fluidic device of Example 3 after 1.841 seconds of contact with a fluid.

FIG. 6C is a stereo microscope image of a portion of the fluidic device of Example 3 after 1.934 seconds of contact with a fluid.

FIG. 6D is a stereo microscope image of a portion of the fluidic device of Example 3 after 2.4021 seconds of contact with a fluid.

While the above-identified figures set forth several embodiments of the disclosure other embodiments are also contemplated, as noted in the description. The figures are not necessarily drawn to scale. In all cases, this disclosure presents the invention by way of representation and not limitation.

### DETAILED DESCRIPTION OF ILLUSTRATIVE EMBODIMENTS

As used herein, the term “essentially free” in the context of a composition being essentially free of a component, refers to a composition containing less than 1% by weight (wt. %), 0.5 wt. % or less, 0.25 wt. % or less, 0.1 wt. % or less, 0.05 wt. % or less, 0.001 wt. % or less, or 0.0001 wt. % or less of the component, based on the total weight of the composition. The term “essentially free” in the context of a feature of a structure (e.g., a surface of a layer), refers to a structure having less than 5% by area of the component, 4% or less, 3% or less, 2% or less, or 1% or less by area of the component, based on the total area of the structure.

As used herein, the term “polymeric” refers to containing at least one polymer.

As used herein, the term “bondable” refers to a material (e.g., a layer) that, after bonding, has an overlap shear of at least 1 megapascals (MPa) or 2 MPa or greater, as measured in accordance with ASTM D-1002-94 at 23-25 degrees Celsius. Each of pressure sensitive adhesives and heat bondable materials are encompassed by the term “bondable”.

As used herein, the term “heat bondable” refers to material (e.g., a layer) that forms a bond to one or more surfaces when heated and the bond formed can be released upon subsequent heating. As opposed to pressure sensitive adhesives, generally, heat-bondable materials have insufficient tack at room temperature to bond to substrates. Unlike thermosetting materials, the bond formed by a heat-bondable material is generally reversible.

As used herein, the term “pressure sensitive adhesive” refers to materials that possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as PSAs include polymers designed and formulated to exhibit the requisite viscoelastic properties resulting in a desired balance of tack, peel adhesion, and shear holding power. PSAs are characterized by being normally tacky at room temperature. Materials that are merely sticky or adhere to a surface do not constitute a PSA: the term PSA encompasses materials with additional viscoelastic properties. PSAs are adhesives that satisfy the Dahlquist criteria for tackiness, which means that the shear storage modulus is typically 3×10⁵ Pa (300 kPa) or less when measured at 25° C. and 1 Hertz (6.28 radians/second). PSAs typically exhibit adhesion, cohesion, compliance, and elasticity at room temperature. As used herein, the term “cavity” refers to an empty space, which is defined by at least one wall of a (e.g., solid) object.

As used herein, the term “chamber” refers to a cavity that is enclosed by at least one additional wall.

As used herein, the term “channel” refers to a passageway that allows gas or liquid to exit a fluidic device.

As used herein, the term “dimensionally stable” refers to the ability of a material (e.g., article or polymeric layer) to maintain its size and shape, even under varying environmental conditions and strains.

As used herein, the term “glass transition temperature” (T_{g}), of a polymer refers to the transition of a polymer from a glassy state to a rubbery state and can be measured using Differential Scanning calorimetry (DSC), such as at a heating rate of 10° C. per minute in a nitrogen stream. When the T_{g} of a monomer is mentioned, it is the T_{g} of a homopolymer of that monomer. The homopolymer must be sufficiently high molecular weight such that the T_{g} reaches a limiting value, as it is generally appreciated that a T_{g} of a homopolymer will increase with increasing molecular weight to a limiting value. The homopolymer is also understood to be substantially free of moisture, residual monomer, solvents, and other contaminants that may affect the T_{g}. A suitable DSC method and mode of analysis is as described in Matsumoto, A. et. al., J. Polym. Sci. A., Polym. Chem. 1993, 31, 2531-2539.

As used herein, the term “Vicat softening temperature” of a polymer refers to the determination of the softening point for a material that has no definite melting point. It is taken as the temperature at which the specimen is penetrated to a depth of 1 mm by a flat-ended needle with a under a specific load.

As used herein, the term “hydrophilic” refers to a surface that is wet by aqueous solutions and does not express whether or not the material absorbs aqueous solutions. By “wet” it is meant that the surface exhibits an advancing (maximum) water contact angle of less than 90°, preferably 45° or less. As used herein, the term “hydrophobic” refers to a surface that exhibits an advancing water contact angle of 90° or greater.

As used herein, the term “fiducial” refers to a structure or mark that provides a fixed basis of comparison.

As used herein, “curing” means the hardening or partial hardening of a composition by any mechanism, e.g., by heat, light, radiation, e-beam, microwave, chemical reaction, or combinations thereof. As used herein, the term “hardenable” refers to a material that can be cured or solidified, e.g., by heating to remove solvent, heating to cause polymerization, chemical crosslinking, radiation-induced polymerization or crosslinking, or the like. As used herein, “cured” refers to a material or composition that has been hardened or partially hardened (e.g., polymerized or crosslinked) by curing.

As used herein, “surface roughness” refers to the smoothness of a material surface and is quantified as “Rₐ”, which refers to the average surface roughness and is defined as the integral of the absolute value of the distance from the mean elevation. The mean elevation is the arithmetic average of the height profile of the surface. The function z(x) refers to the difference between the height and the mean elevation at a position x measured over an evaluation length l:

$${R}_{a} = \frac{1}{l} {\int }_{0}^{l} \vert z ( x ) \vert dx$$

The term “R_{q}” represents the root mean square value of the ordinate values z(x) within the sampling length l

$${R}_{q} = \sqrt{\frac{1}{l} {\int }_{0}^{l} {z}^{2} ( x ) dx}$$

The term “Rₛₖ” refers to the quotient of the mean cube value of the ordinate values z(x) and the cube of R_{q} within the sampling length l

$${R}_{sk} = \frac{1}{{R}_{q}^{3}} [ \frac{1}{l} {\int }_{0}^{l} {z}^{3} ( x ) dx ]$$

The elevation can be measured using an optical profilometer (e.g., a Wyko NT3300 optical profilometer from Veeco Instruments Inc., Plainview, New Jersey).

As used herein, “substantially planar” with respect to a layer means that a surface of the layer is essentially free of recesses and/or protrusions extending above and/or below a plane of the layer, the recesses and/or protrusions having a depth or height of greater than 5 micrometers (μm), 4.75 μm, 4.5 μm, 4.25 μm, 4 μm, 3.75 μm, 3.5 μm, 3.25 μm, 3 μm, 2.75 μm, 2.5 μm, 2.25 μm, 2 μm, 1.75 μm, 1.5 μm, 1.25 μm, 1 μm, 750 nm, 600 nm, 500 nm, 400 nm, 300 nm, or greater than 200 nm. Typically, recesses and/or protrusions have a depth or height of 5 nm or greater, 10 nm, 15 nm, 20 nm, 25 nm, 30 nm, 40 nm, 50 nm, 75 nm, 100 nm, 125 nm, or 150 nm or greater. In some cases, deviations from planarity are caused by the thickness of a hydrophilic mask. Typically, protrusions on the surface lack an engineered pattern, but rather tend to be random. The depth or height of a recesses or protrusion present on a layer surface can be measured with a confocal microscope.

As used herein, “thermoplastic” refers to a polymer that flows when heated sufficiently above its glass transition point and become solid when cooled.

As used herein, “thermoset” refers to a polymer that permanently sets upon curing and does not flow upon subsequent heating. Thermoset polymers are typically crosslinked polymers.

As used herein, “transparent” refers to a material (e.g., a layer) that has at least 50% transmittance, 70% transmittance, or optionally greater than 90% transmittance over at least the 400 nanometer (nm) to 700 nm portion of the visible light spectrum.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

In this application, terms such as “a”, “an”, and “the” are not intended to refer to only a singular entity but include the general class of which a specific example may be used for illustration. The terms “a”, “an”, and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties). The term “substantially”, unless otherwise specifically defined, means to a high degree of approximation (e.g., within +/−10% for quantifiable properties) but again without requiring absolute precision or a perfect match. Terms such as same, equal, uniform, constant, strictly, and the like, are understood to be within the usual tolerances or measuring error applicable to the particular circumstance rather than requiring absolute precision or a perfect match.

Fluidic devices according to at least certain embodiments of the present disclosure provide a multilayered, melt bondable, spontaneous capillary microfluidic article that may be useful, for instance, for liquid sample acquisition. Suitable applications include, for example, bodily fluid acquisition for diagnostic devices (e.g., blood glucose strips) and medical wearables (e.g., sweat sensors). For instance, blood glucose test strips are a type of disposable point of care sensor designed to analyze small volumes of blood, for instance from a stick to a person's finger. The strips conventionally include a bottom sensor layer laminated to multiple film layers configured to form a small chamber for the blood to enter the strip and be exposed to the sensor chemistry.

It has been discovered that certain molding processes are capable of generating planar polymeric layers with bondable regions to overcome at least one limitation of current methods of making fluidic devices. The bond forms a hermetic seal between the polymeric layers, whereas the hydrophilic patterned region does not bond. It was also discovered that, for instance, during heat-bonding, melted polymer did not break through the hydrophilic pattern region to result in bonding in an undesired region. Advantageously, the bonding process can be adapted to minimize inactivation of any sensor chemistry present (e.g., reagent(s)) while achieving high adhesion strength. Incorporation of these features in a structure according to at least certain embodiments of the present disclosure simplifies device assembly by reducing the number of input materials required to form a single mechanically robust fluidic device.

In a first aspect, the present disclosure provides a fluidic device. The fluidic device comprises:
a) a first bondable polymeric layer having a first major surface that is substantially planar;
b) a second polymeric layer having a first major surface that is substantially planar; and
c) a hydrophilic mask material disposed on a first portion of the first major surface of the first bondable polymeric layer, wherein a surface of the hydrophilic mask material exhibits an advancing contact angle with water of less than 90 degrees;
wherein a second portion of the first major surface of the first bondable polymeric layer is bonded to a first portion of the first major surface of the second polymeric layer, wherein the hydrophilic mask material and a second portion of the first major surface of the second polymeric layer are in direct contact with each other at at least one point, wherein an open volume is defined by interstitial space located between the hydrophilic mask material and the second portion of the first major surface of the second polymeric layer, wherein the open volume comprises two or more openings and wherein at least one of the openings is located at an edge of the first bondable polymeric layer. Typically, the interstitial space comprises a volume of 500 nanoliters or less per square centimeter of the first portion of the first major surface of the first bondable polymeric layer, such as 450 nanoliters, 400 nanoliters, 350 nanoliters, 300 nanoliters, or even 250 nanoliters or less; and 0.5 nanoliters or more, 1 nanoliter, 2 nanoliters, 5 nanoliters, 7 nanoliters, 10 nanoliters, 15 nanoliters, 20 nanoliters, 25 nanoliters, 30 nanoliters, 35 nanoliters, 40 nanoliters, 45 nanoliters, 50 nanoliters, 55 nanoliters, 60 nanoliters, 70 nanoliters, 80 nanoliters, 90 nanoliters, or 100 nanoliters or more. This is advantageous in allowing use of a much smaller fluid sample with fluidic devices according to the present disclosure than devices including a cavity into which fluid flows.

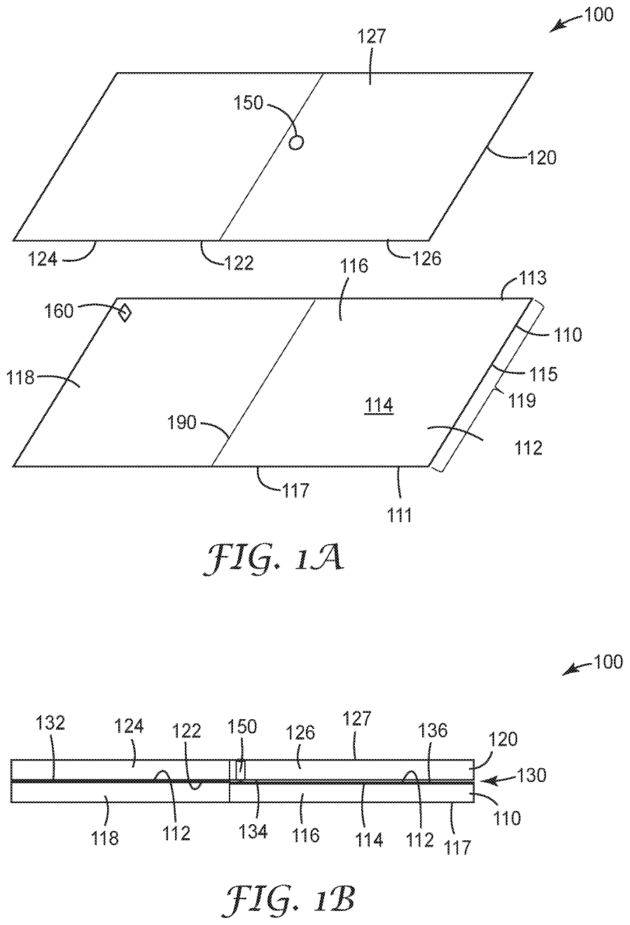

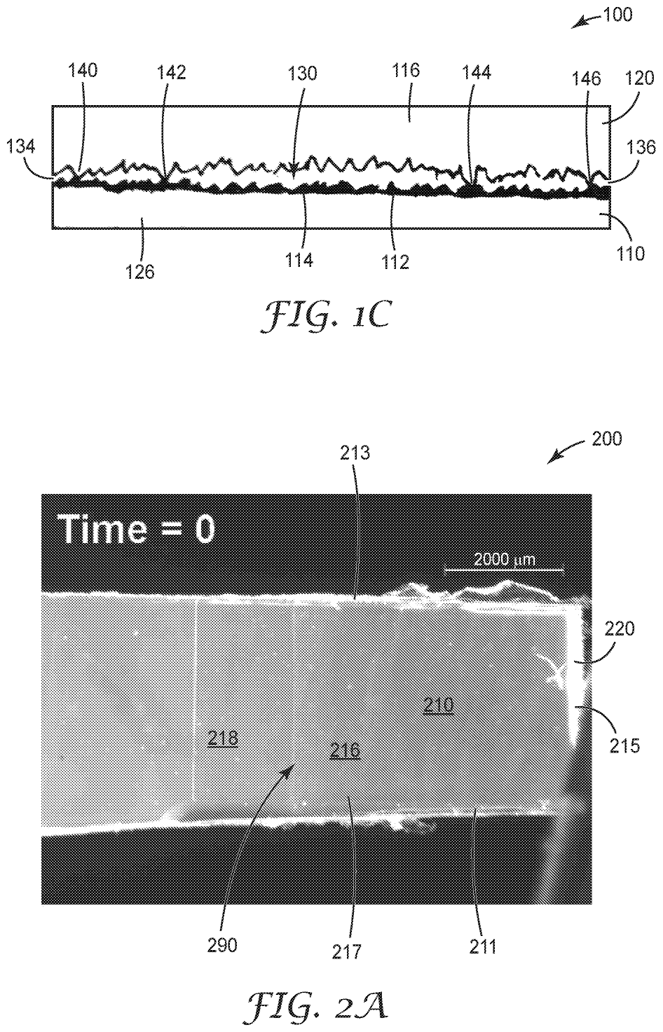

Referring to FIGS. 1A-1B, general schematic illustrations are provided of an exploded view and a cross-sectional view, respectively, of a fluidic device 100. The fluidic device 100 comprises a first bondable polymeric layer 110 having a first major surface 112 that is substantially planar, and a hydrophilic mask material 114 disposed on a first portion 116 of the first major surface 112 of the first bondable polymeric layer 110. The fluidic device 100 further comprises a second polymeric layer 120 having a first major surface 122 that is substantially planar. A second portion 118 of the first major surface 112 of the first bondable polymeric layer 110 is bonded 132 to a first portion 124 of the first major surface 122 of the second polymeric layer 120.

In this embodiment, the first portion 116 of the first major surface 112 of the first bondable polymeric layer 110 on which the hydrophilic mask material 114 is disposed is a continuous area that extends from a first edge 111 of the first bondable polymeric layer 110 to each of an opposing second edge 113 of the first bondable polymeric layer 110 and a third edge 115 of the first bondable polymeric layer 110 that is located between the first edge 111 and the second edge 113 of the first bondable polymeric layer 110, and to an interface 190 between the first portion 116 and the second portion 118. Optionally, the first portion 116 extends along an entirety 119 of the third edge 115. For instance, in this embodiment, the first portion 116 of the first major surface 112 has a continuous quadrilateral shape, bounded by the interface 190, the first edge 111, the second edge 113, and the third edge 115.

Referring to FIG. 1C, a schematic illustration is provided of a portion of a cross-section of a fluidic device 100, wherein the hydrophilic mask material 114 and a second portion 126 of the first major surface 122 of the second polymeric layer 120 are in direct contact with each other at at least one point, in this case four points: 140, 142, 144, 146. An open volume 130 is defined by interstitial space located between the hydrophilic mask material 114 and the second portion 126 of the first major surface 122 of the second polymeric layer 120 (see also FIG. 1B for such an open volume 130). The open volume 130 comprises two or more openings 134, 136 and at least one of the openings 134, 136 is located at an edge (any of edges 111, 113, and/or 115 shown in FIG. 1A) of the first bondable polymeric layer 110. As such, there is not intimate contact between the hydrophilic mask material 114 and the second portion 126 of the first major surface 122 of the second polymeric layer 120 throughout the whole nonbonded area (e.g., across the entirety of the first portion 116 of the first bondable polymeric layer 110), but rather at certain points in the area. In some embodiments of fluidic devices, there is a gap between a first edge of the first bondable polymeric layer and a directly adjacent first edge of the second polymeric layer of up to 5 μm (see, e.g., at openings 134 and/or 136 in FIG. 1C).

In any fluidic device according to the present disclosure, at least one of the first portion of the first major surface of the first bondable polymeric layer or the second portion of the first major surface of the second polymeric layer may exhibit an average surface roughness (Rₐ) of 1 nanometer (nm) to 5 micrometers (μm). For instance, a major surface may exhibit an average surface roughness (Rₐ) of 1 nm or greater, 2 nm, 5 nm, 10 nm, 15 nm, 20 nm, 25 nm, 30 nm, 35 nm, 40 nm, 45 nm, 50 nm, 75 nm, 100 nm, 125 nm, 150 nm, 175 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, or 1 μm or greater; and 5 μm or less, 4.75 μm, 4.5 μm, 4.25 μm, 4 μm, 3.75 μm, 3.5 μm, 3.25 μm, 3 μm, 2.75 μm, 2.5 μm, 2.25 μm, 2 μm, 1.75 μm, 1.5 μm, 1.25 μm, 1 μm, 750 nm, 500 nm, or 250 nm or less. In the embodiment of FIG. 1C, both the hydrophilic mask material 114 on the first portion 116 of the first major surface 112 of the first bondable polymeric layer 110 and the second portion 126 of the first major surface 122 of the second polymeric layer 120 have rough surfaces and may exhibit an average surface roughness (Rₐ) within the ranges described above. In some cases, the at least one point of contact between the hydrophilic mask material and the second portion of the first major surface occurs at a location of a maximum height of the surface roughness, for instance at point 144 in FIG. 1C.

The first bondable polymeric layer of the embodiment of FIG. 1A further comprises a fiducial mark 160, namely a pre-defined shaped mark located on the first major surface 112 of the first bondable polymeric layer 110. A fiducial structure or mark advantageously provides a reference mark for improved alignment of the article during further manufacturing and/or processing steps. Optionally, the first bondable polymeric layer, the second polymeric layer, or both comprises a fiducial structure or mark. In certain embodiments, instead of a mark, a fiducial structure may be provided on the fluidic device. A fiducial structure, for instance, may be a three-dimensional structure formed on the fluidic device (e.g., during formation of the first or second polymeric layer).

In any fluidic device according to the present disclosure, a channel may be provided, connecting the first portion of the first major surface of the first bondable polymeric layer with at least one of a second major surface of the first bondable polymeric layer or a second major surface of the second polymeric layer. The channel is a passageway that allows gas or liquid to exit the fluidic device, and may be helpful in wicking fluid into the fluidic device as displaced gas or liquid can (e.g., more) readily exit the fluidic device, through the channel. In the embodiment shown in FIGS. 1A and 1B, the channel 150 has a generally cylindrical shape: however, the shape is not particularly limited. A generally cylindrical channel can be formed, for example, using laser drilling through at least one of the first bondable polymeric layer 110 or the second polymeric layer 120.

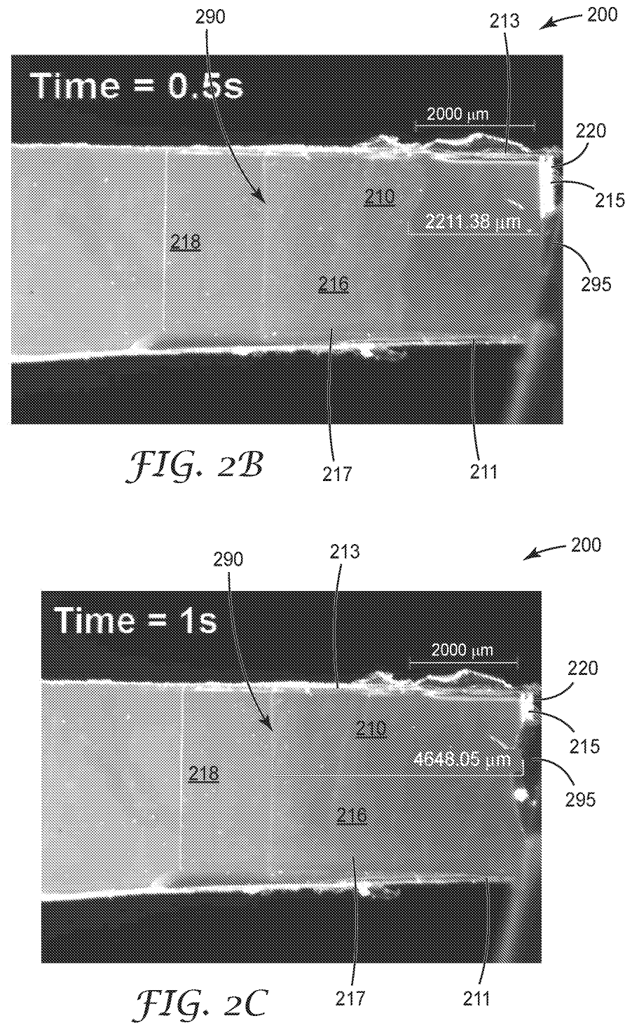

Referring now to FIGS. 2A-2C, microscopy images are provided of a portion of an exemplary fluidic device of Example 4 (described below) prior to contact with a fluid (FIG. 2A), after 0.5 seconds of contact with a fluid (FIG. 2B) and after 1 second of contact with a fluid (FIG. 2C). FIG. 2A provides an image of a portion of a fluidic device 200 having a first bondable polymeric layer 210 having a hydrophilic mask material (not shown) disposed on a first major surface (not shown) and an opposing second major surface 217. The first bondable polymeric layer 210 is bonded to a second polymeric layer 220 in the second portion 218 of the first bondable polymeric layer 210 but not in the first portion 216 of the first bondable polymeric layer 210. In this embodiment, the first portion 216 is a continuous portion that extends fully along a first edge 211, along an opposing second edge 213, and along a third edge 215 located between the first edge 211 and the second edge 213. This configuration allows for venting of air and/or liquid at any point along each of the edges during use of the fluidic device 200.

A 3 microliter sample of human blood 295 was contacted with the third edge 215 of the fluidic device 200 and FIG. 2B shows that after half a second from the time of contact, a portion of the sample of the blood traveled a distance of at least 2000 micrometers into the fluidic device 200 from the third edge 215 (i.e., within the open volume formed by interstitial space between the unbonded hydrophilic mask material and the second polymeric layer 220). The distance traveled can be seen by the presence of the darker shade of grey provided by the presence of the blood 295 and marked at one point along the leading edge of the blood 295 by a bracket labeled 2211.38 μm. FIG. 2C shows that after one second from the time of contact, a portion of the blood 295 traveled the full distance into the fluidic device 200 from the third edge 215 to the interface 290 laterally through the open volume, where the first portion 216 of the first bondable polymeric layer 210 (i.e., including the hydrophilic mask material) meets the second portion 218 of the first bondable polymeric layer 210 (i.e., bonded to the second polymeric layer 220). The interface is located along the vertical line to which the 290 arrow points and is marked by a bracket labeled 4648.05μ m.

Advantageously, the use of a hydrophilic mask material allows for the formation of intricate fluidic designs not easily manufactured through microreplication, embossing, or die cutting. A design or pattern is not particularly limited and may be configured in various ways. For instance, in any fluidic device according to the present disclosure, in the first portion of the first major surface of the first bondable polymeric layer on which the hydrophilic mask material is disposed is a pattern that defines a fluid path from:
a) a first portion of the third edge of the first bondable polymeric layer to i) at least one of the first edge or the second edge of the first bondable polymeric layer or ii) back to the third edge of the first bondable polymeric layer at a location spaced apart from the first portion of the third edge;
b) a first portion of the second edge of the first bondable polymeric layer to i) at least one of the first edge or the third edge of the first bondable polymeric layer or ii) back to the second edge of the first bondable polymeric layer at a location spaced apart from the first portion of the second edge; or
c) a first portion of the first edge of the first bondable polymeric layer to i) at least one of the second edge or the third edge of the first bondable polymeric layer or ii) back to the first edge of the first bondable polymeric layer at a location spaced apart from the first portion of the first edge.

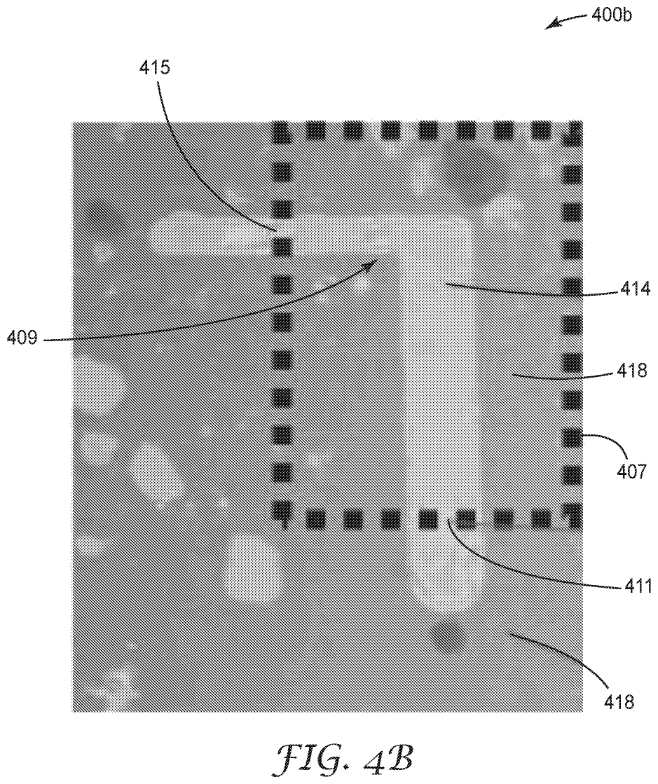

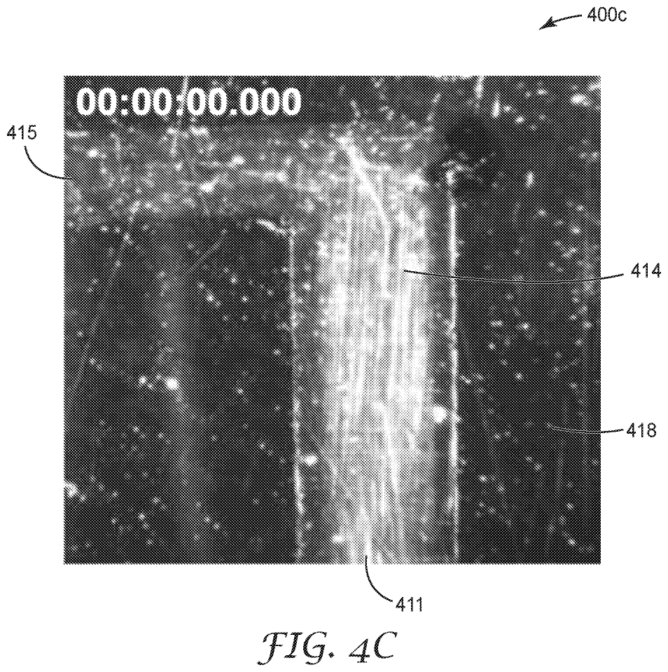

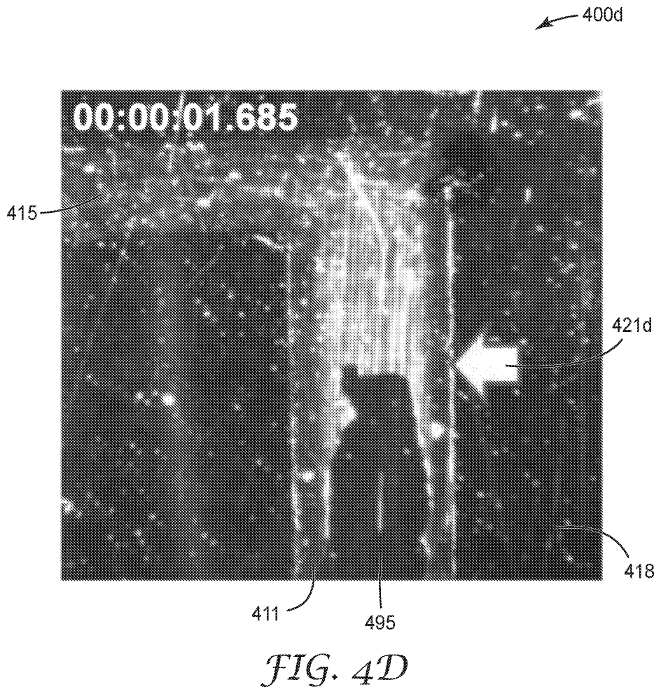

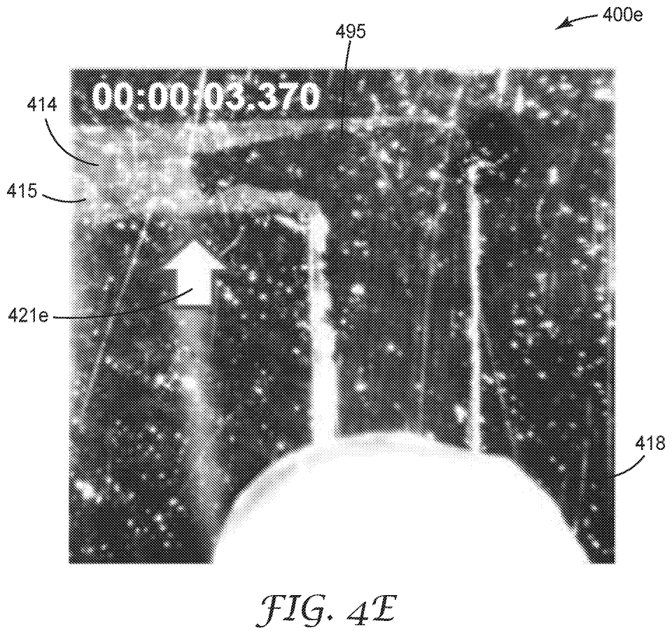

As such, referring again to FIG. 1A, to provide at least two openings in the fluidic device, a pattern (not shown) of hydrophilic mask material 114 may be applied to the first portion 116 of the first major surface 112 of the first bondable polymeric layer 110 that defines a fluid path starting from a location along any one of the first edge 111, the second edge 113, or the third edge 115. Moreover, the fluid path may extend to a separate location of the same edge from which it started or may extend to a location on either of the other two edges. There is no alignment between any pattern on the second polymeric layer (e.g., deviations from planarity such as surface roughness) and a pattern of the hydrophilic mask material. Some specific suitable hydrophilic mask material patterns are shown in FIG. 4A and described in detail below.

Referring to FIG. 4A, a photograph is provided of tapes comprising three different patterned masks 400, 500, and 600. In the patterned mask 400, the pattern is designed such that the first portion of the first major surface of the first bondable polymeric layer will have a shape that has a length that is at least 10 times greater than a width of the shape. Such a shape can be imparted by the open area 403 (i.e., having a width of 1 mm and a length of 10 mm), which is connected to the open area 405 by a 90 degree angle. This pattern is designed such that a fluid wicks from one edge to a different (e.g., adjacent) edge of a fluidic device.

In the patterned mask 500, the pattern is designed such that the first portion of the first major surface of the first bondable polymeric layer will have a linear shape that comprises a straight portion. In other cases, a linear shape is provided that comprises a curved portion, or combinations of a straight portion and a curved portion. This pattern is designed such that a fluid wicks from one edge to a different (i.e., opposing) edge of a fluidic device. In the patterned mask 600, the pattern has a rectangular design such that the first portion of the first major surface of the first polymeric layer will have a shape essentially the same as in FIG. 2A (e.g., a continuous portion that extends fully along a first edge, along an opposing second edge, and along a third edge located between the first edge and the second edge).

Suitable hydrophilic mask materials include for instance and without limitation, plasma deposited silicon/oxygen materials or diamond like glass, a nanostructure (e.g., such as using the methods described in U.S. Pat. Nos. 8,634,146 and 10,134,566 (each to David et al.)), a surfactant, a polyester, a polyamide, a polyurethane, a poly(vinyl alcohol), a poly(alkylene glycol), a poly(alkylene oxide), a poly(vinyl pyrrolidone), a rubber elastomer, or any combination thereof.

To prepare a (bonded) fluidic device, the first bondable polymeric layer includes for instance and without limitation, a low density polyethylene, ethylene vinyl acetate, ethylene acrylic acid, a polyurethane, copolymers of a polyester and a polyolefin, copolymers of a polyurethane and an aromatic poly(meth)acrylate, copolymers of a polycaprolactone and a polyurethane, or a combination thereof. One suitable commercially available polyurethane if from Lubrizol (Wickliffe, OH) under the trade designation “PEARLBOND 1160L”. A suitable commercially available ethylene vinyl acetate (EVA) is from The Dow Chemical Company under the trade designation “DUPONT ELVAX 3180”. A suitable commercially available ethylene acrylic acid is from SK Global Chemical, Seoul South Korea under the trade designation “PRIMACOR 3330”. Further, various additives may be included in the first bondable polymeric layer, for example plasticizers, antioxidants, pigments, release agents, antistatic agents, and the like.

In any embodiment, the average thickness of the first bondable layer is 5 micrometers or more, 7.5 micrometers, 10 micrometers, 12.5 micrometers, 15 micrometers, 17.5 micrometers, or 20 micrometers or more; and 50 micrometers or less, 45 micrometers, 40 micrometers, 35 micrometers, 30 micrometers, or 25 micrometers or less. Stated another way, the first bondable polymeric layer may have an average thickness of 5 micrometers to 50 micrometers. The average thickness may be determined by measuring the thickness in at least 5 places, located at least 0.5 millimeters apart from each other, and taking the average of all of the measured thicknesses.

In some cases, suitable polymeric materials for the first bondable polymeric layer include for instance and without limitation, a low density polyethylene, ethylene vinyl acetate, a polyurethane, copolymers of a polyester and a polyolefin, copolymers of a polyurethane and an aromatic poly(meth)acrylate, copolymers of a polycaprolactone and a polyurethane, or a combination thereof. Further, various additives may be included in the first bondable polymeric layer, for example plasticizers, antioxidants, pigments, release agents, antistatic agents, and the like.

Suitable polymeric materials for the second polymeric layer include for instance and without limitation, a polyolefin (e.g., high density polyethylene (HDPE), medium density polyethylene (MDPE), or low density polyethylene (LDPE)), a polyester, a polyamide, a poly(vinyl chloride), a polyether ester, a polyimide, a polyesteramide, a polyacrylate, a polyvinylacetate, or a hydrolyzed derivative of polyvinylacetate. In certain embodiments, polyolefins are preferred because of their excellent physical properties, ease of processing (e.g., replicating the surface of a tool), and typically low cost. Also, polyolefins are generally tough, durable and hold their shape well, thus being easy to handle after article formation. In select embodiments, the second polymeric layer includes the polyester polyethylene terephthalate (PET). One suitable commercially available PET is a 5 mil (127 micrometer) thick PET sheet from Tekra (New Berlin, WI) under the trade designation “MELINEX 454”. A suitable commercially available LDPE is from The Dow Chemical Company (Midland Michigan) under the trade designation “DOW 955I LDPE”.

A surface treatment can be applied to a surface of the second polymeric layer to improve bonding with the first bondable polymeric layer. Suitable surface treatments can include flame treatment, corona treatment, a metallic primer (e.g., layer of a metal such as gold), or a chemical primer.

Preferably, the second polymeric layer has a maximum thickness of 500 micrometers, 475 micrometers, 450 micrometers, 425 micrometers, 400 micrometers, 375 micrometers, 350 micrometers, 325 micrometers, 300 micrometers, 275 micrometers, 250 micrometers, 225 micrometers, 200 micrometers, or 175 micrometers; and a minimum thickness of 50 micrometers, 75 micrometers, 100 micrometers, 125 micrometers, or 150 micrometers.

In certain embodiments, the first bondable polymeric layer has a Vicat softening temperature (T_{g}) of 100 degrees Celsius (° C.) or less, 95° C., 90° C., 85° C., 80° C., 75° C., or 70° C. or less; and 45° C. or more, 50° C., 55° C., 60° C., or 65° C. or more. The Vicat softening temperature of the first bondable polymeric layer is often at least 10% lower than the Vicat softening temperature of the second polymeric layer, 15% lower, 20% lower, 25% lower, 30% lower, 35% lower, or at least 40% lower than the Vicat softening temperature of the second polymeric layer. In certain embodiments, the second polymeric layer has a Vicat softening temperature (T_{g}) of 150 degrees Celsius (C) or less, 145° C., 140° C., 130° C., 120° C., 115° C., or 110° C. or less; and 65° C. or more, 70° C., 75° C., 80° C., or 85° C. or more. Using polymeric layers with different Vicat softening temperatures may assist in heat bonding the second polymeric layer to the first bondable polymeric layer while each polymeric layer maintains its substantially planar first major surface.

In some embodiments, the first bondable polymeric layer, the second polymeric layer, or both, are transparent to visible light (as defined above). Providing one or more transparent layers can be advantageous for certain applications in which a sample reaction can be optically detected through at least a portion of the article.

In preferred embodiments, the article is advantageously dimensionally stable with a strain less than 50%, 40%, 30%, 20%, 15%, 10%, or less than 5%, at a temperature of 25 degrees Celsius (° C.). As used herein, “strain” refers to a stretch ratio or an extension ratio. It is defined as the ratio between the final length l and the initial length L of the material line in any particular direction. For instance, an elongation of 50%=1.5L. Dimensional stability helps to resist deformation of the fluidic device when the device is handled, decreasing the likelihood of damaging the structure before or during use.

Fluidic devices according to at least certain embodiments of the present disclosure are capable of spontaneously and uniformly transporting liquids (e.g., water, urine, blood, or other aqueous solutions) along the open volume between the hydrophilic mask material and a second portion of the first major surface of the second polymeric layer, from a first opening with which the liquid is contacted towards at least a second opening. This capability is often referred to as wicking. Two general factors that influence the ability of layers to spontaneously transport liquids are (i) the structure or topography of the surface (e.g., capillarity, shape of a cavity) and (ii) the nature of the surface (e.g., surface energy). As the first and second polymeric layers are each substantially planar and directly adjacent to each other, to achieve a specific desired amount of fluid transport capability a designer may adjust the surface energy of the polymeric layer surface upon which the hydrophilic masking material is disposed. In order to achieve wicking, the surface of that polymeric layer must be capable of being “wet” by the liquid to be transported, which is achieved using the hydrophilic mask material. Generally, the susceptibility of a solid surface to be wet by a liquid is characterized by the contact angle that the liquid makes with the solid surface after being deposited on a horizontally disposed surface and allowed to stabilize thereon. This angle is sometimes referred to as the “static equilibrium contact angle,” and sometimes referred to herein merely as “advancing contact angle.” As defined above, a material is hydrophilic if it has an advancing contact angle of less than 90 degrees.

Hydrophilicity can be achieved through one or more of material selection, additives included in a material, or surface treatment. Often, the hydrophilic mask material has an average thickness (e.g., of the material disposed on the first or second polymeric layer) of 1 nm to 1 μm. For instance, the hydrophilic mask material may have an average thickness of 1 nm or greater, 2 nm, 5 nm, 10 nm, 15 nm, 20 nm, 25 nm, 30 nm, 35 nm, 40 nm, 45 nm, 50 nm, 75 nm, 100 nm, 125 nm, 150 nm, 175 nm, 200 nm, 250 nm, 300 nm, 350 nm, 400 nm, 450 nm, or 500 nm or greater; and 1 μm or less, 950 nm, 900 nm, 850 nm, 800 nm, 750 nm, 700 nm, 650 nm, 600 nm, 550 nm, 500 nm, 450 nm, 400 nm, 350 nm, 300 nm, or 250 nm or less.

In some embodiments, the hydrophilic mask material includes a surfactant, a surface structure, a surface treatment, a hydrophilic polymer, or a combination thereof. Suitable surfactants include for instance and without limitation, C8-C18 alkane sulfonates: C8-C18 secondary alkane sulfonates; alkylbenzene sulfonates: C8-C18 alkyl sulfates; alkylether sulfates; sodium laureth 4 sulfate; sodium laureth 8 sulfate; dioctylsulfosuccinate, sodium salt; lauroyl lacylate; stearoyl lactylate; or any combination thereof. One or more surfactants can be applied by conventional methods, such as by wiping a coating of the surfactant on the surface of the second polymeric layer and allowing the coating to dry. A suitable surface structure includes a discontinuous coating comprising pillars and interstitial space between the pillars. Nanopillars may be formed using methods described in, e.g., U.S. Pat. Nos. 8,634,146 and 10,134,566 (each to David et al.). A suitable surface treatment includes a hydrophilic coating comprising plasma deposited silicon/oxygen materials and/or diamond-like glass (DLG) materials. Plasma deposition of each of silicon/oxygen materials and DLG material is described, for instance, in PCT Publication No. WO 2007/075665 (Somasiri et al.). Further, examples of suitable DLG materials are disclosed in U.S. Pat. No. 6,696,157 (David et al.), U.S. Pat. No. 6,881,538 (Haddad et al.), and U.S. Pat. No. 8,664,323 (Iyer et al.). Suitable hydrophilic polymers include for instance and without limitation, a polyester, a polyamide, a polyurethane, a poly(vinyl alcohol), a poly(alkylene glycol), a poly(alkylene oxide), a poly(vinyl pyrrolidone), a rubber elastomer, or any combination thereof.

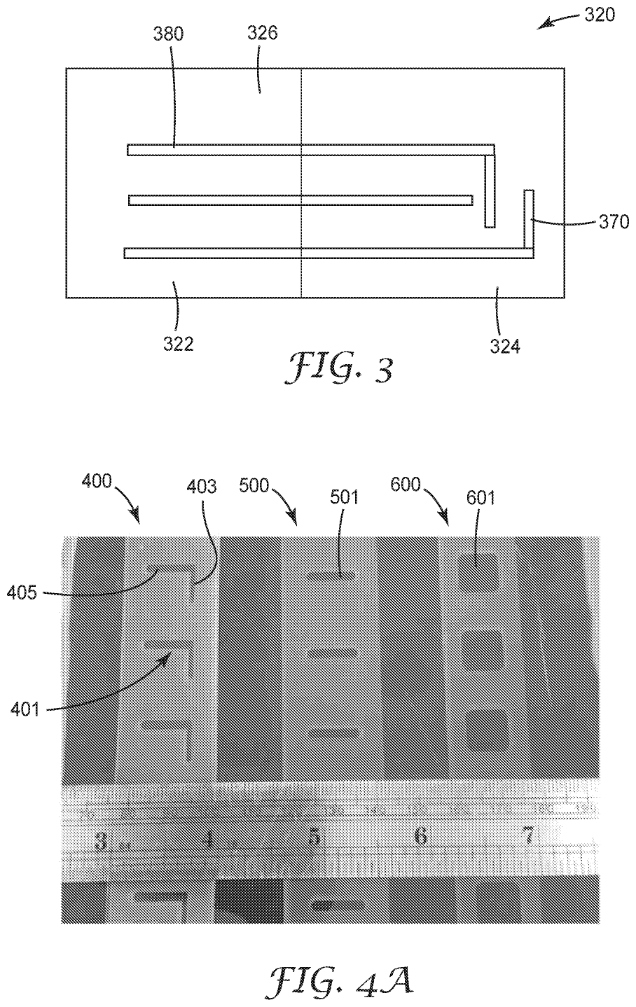

In any fluidic device according to the present disclosure, at least one of the first bondable polymeric layer or the second polymeric layer may comprise a reagent, for instance a reagent disposed on a major surface of the layer. The reagent is preferably configured to react with a sample and provide at least one response selected from the response types of electrochemical, optical, fluorescent, and/or chemiluminescent. Some suitable reagents include for instance and without limitation, fluorogenic or chromogenic indicators, electrochemical reagents, agglutination reagents, analyte specific binding agents, amplification agents such as enzymes and catalysts, photochromic agents, dielectric compositions, analyte specific reporters such as enzyme-linked antibody probes, DNA probes, RNA probes, fluorescent or phosphorescent beads, or any combination thereof. When a layer includes a reagent, that layer often further comprises a detector. For instance, referring to FIG. 3, a generalized schematic top view illustration is provided of second polymeric layer 320 comprising a detector 370 disposed on a first major surface 322 of the second polymeric layer 320, including a reagent (not shown). In select embodiments, the detector 370 comprises an electrode 380 also disposed on a first major surface 122 of the second polymeric layer 320. In such embodiments, the open volume of the fluidic device (e.g., over the first portion 324 of the second polymeric layer 320) is in fluid communication with the electrode. Alternatively, such optional detector and electrode may be provided on the first bondable polymeric layer. For a device application of a blood glucose test strip, for instance, blood from a finger prick enters an opening of the fluidic device, contacts the reagent, and a response from the reaction of the reagent with the blood is measured by the detector.

### Methods

Fluidic devices according to the present disclosure may be formed by a method comprising:
a) applying a hydrophilic mask material to a first portion of a first major surface of a first polymer;
b) disposing a second polymer on the first polymer; and
c) applying compression to the first polymer and the second polymer at an elevated temperature to bond a second portion of the first major surface of the first bondable polymeric layer to the second polymeric layer to form the fluidic device.

Often, the first polymer and the second polymer are bonded together (in step c)) using a tool having a substantially planar major surface. The hydrophilic mask material is typically applied by placing a mask over the second portion of the first major surface of the first bondable polymeric layer to block application of the hydrophilic mask material, followed by depositing the hydrophilic mask material onto the first portion of the first major surface of the first bondable polymeric layer. Masks are known to those of skill in the art for assisting in covering only desired regions of a surface with a material by obstructing the regions on which it is not desired to apply the hydrophilic mask material. Alternatively, the hydrophilic mask material may be applied by printing the material in a pattern, for instance using a flexographic printing process.

The first polymer and the second polymer are as described above in detail with respect to the materials for the first bondable polymeric layer and the second polymeric layer, respectively, of the fluidic device. In some embodiments, the first polymer and the second polymer are independently provided in the form of a sheet (e.g., layer) or as particulates (e.g., pellets). The elevated temperature used in certain embodiments of the method is 300 degrees Fahrenheit (° F.) or less, 290° F., 285° F., 280° F., 275° F., 270° F., 265° F., 260° F., 255° F., 250° F., 245° F., 240° F., 235° F., 230° F., or 225° F. or less; and 150° F. or greater, 155° F., 160° F., 165° F., 170° F., 175° F., 180° F., 185° F., 190° F., 195° F., or 200° F. or greater. Applying compression optionally further includes cooling the fluidic device following exposure to the elevated temperature, such as by allowing to cool by exposure to ambient temperature, or by actively cooling the tool and/or the fluidic article.

In some embodiments, the method includes applying compression for 10 minutes or less, 9 minutes, 8 minutes, 7 minutes, 6 minutes, 5 minutes, or 4 minutes or less; and 0.5 minutes or greater, 1 minute, 2 minutes, or 3 minutes or greater. The method may include applying the compression to the entire area at a pressure of 15,000 pounds or less, 14,000 pounds, 13,000 pounds, 12,500 pounds, 12,000 pounds, 11,000 pounds, 10,000 pounds, 9,000 pounds, 8,000 pounds, or 7,500 pounds or less; and 3,000 pounds or more, 3,500 pounds, 4,000 pounds, 4,500 pounds, 5,000 pounds, 5,500 pounds, 6,000 pounds, 6,500 pounds, or 7,000 pounds. For instance, when applying the compression at 10,000 pounds to an area of 81 square inches (522.58 square centimeters), the compression would be 12 psi (0.083 megaPascals).

In select embodiments, the method further includes subjecting the fluidic device to post-curing, which can be performed using actinic radiation, such as UV radiation, e-beam radiation, visible radiation, or any combination thereof. The skilled practitioner can select a suitable radiation source and range of wavelengths for a particular application without undue experimentation. So called post cure ovens, which combine UV radiation and thermal energy, are particularly well suited for use in the post-cure processes. In general, post-curing improves the mechanical properties and stability of the article relative to the same article that is not post cured.

At least certain embodiments of the present methods unexpectedly provide multilayered spontaneous capillary microfluidic devices having small open volumes between substantially planar surfaces. In contrast, PCT Publication WO 2020/261086 (Halverson et al.) describes the incorporation of a cavity formed in an article, which is not needed in the present fluidic devices to achieve capillary flow of a fluid through a device. Further, each of PCT Publication WO 98/45693 (Soane et al.) and U.S. Pat. No. 7,553,393 (Derand et al.) describe the use of heat-sealing cover layers to microfluidic structures formed in a second film. These documents, however, do not address the situation where the heat bond is located on the cavity side (e.g., channels of the microfluidic structures), as would be required for lamination to a detection (e.g., sensor) layer. Each of U.S. Pat. No. 5,798,031 (Charlton et al.) and U.S. Pat. No. 8,617,367 (Edelbrock et al.) describe thermoforming features in a heat bondable film for subsequent lamination to a detection layer. In these examples, the film is placed with the heat bondable layer facing away from the thermoforming tool to prevent it from adhering to the tool surface during thermoforming. The result of this orientation requirement is the generation of films having a Z axis profile, which are less mechanically robust than articles having at least one planar major surface.

### Select Embodiments of the Disclosure

In a first embodiment, the present disclosure provides a fluidic device. The fluidic device comprises a) a first bondable polymeric layer having a first major surface that is substantially planar; b) a second polymeric layer having a first major surface that is substantially planar; and c) a hydrophilic mask material disposed on a first portion of the first major surface of the first bondable polymeric layer. A surface of the hydrophilic mask material exhibits an advancing contact angle with water of less than 90 degrees. A second portion of the first major surface of the first bondable polymeric layer is bonded to a first portion of the first major surface of the second polymeric layer. The hydrophilic mask material and a second portion of the first major surface of the second polymeric layer are in direct contact with each other at at least one point. An open volume is defined by interstitial space located between the hydrophilic mask material and the second portion of the first major surface of the second polymeric layer. The open volume comprises two or more openings and at least one of the openings is located at an edge of the first bondable polymeric layer.

In a second embodiment, the present disclosure provides a fluidic device according to the first embodiment, wherein the first portion of the first major surface of the first bondable polymeric layer on which the hydrophilic mask material is disposed is a continuous area that extends from a first edge of the first bondable polymeric layer to each of an opposing second edge of the first bondable polymeric layer and a third edge of the first bondable polymeric layer that is located between the first edge and the second edge of the first bondable polymeric layer.

In a third embodiment, the present disclosure provides a fluidic device according to the second embodiment, wherein the first portion extends along an entirety of the third edge.

In a fourth embodiment, the present disclosure provides a fluidic device according to the first embodiment, wherein the first portion of the first major surface of the first bondable polymeric layer on which the hydrophilic mask material is disposed is a pattern that defines a fluid path from: a) a first portion of the third edge of the first bondable polymeric layer to i) at least one of the first edge or the second edge of the first bondable polymeric layer or ii) back to the third edge of the first bondable polymeric layer at a location spaced apart from the first portion of the third edge; b) a first portion of the second edge of the first bondable polymeric layer to i) at least one of the first edge or the third edge of the first bondable polymeric layer or ii) back to the second edge of the first bondable polymeric layer at a location spaced apart from the first portion of the second edge; or c) a first portion of the first edge of the first bondable polymeric layer to i) at least one of the second edge or the third edge of the first bondable polymeric layer or ii) back to the first edge of the first bondable polymeric layer at a location spaced apart from the first portion of the first edge.

In a fifth embodiment, the present disclosure provides a fluidic device according to the fourth embodiment, wherein the first portion of the first major surface of the first bondable polymeric layer and the second portion of the first major surface of the first bondable polymeric layer interpenetrate with each other.

In a sixth embodiment, the present disclosure provides a fluidic device according to the fourth embodiment or the fifth embodiment, wherein the first portion of the first major surface of the first bondable polymeric layer has a shape that has a length that is at least 10 times greater than a width of the shape.

In a seventh embodiment, the present disclosure provides a fluidic device according to any of the fourth through sixth embodiments, wherein the first portion of the first major surface of the first bondable polymeric layer has a linear shape that comprises a straight portion, a curved portion, or combinations thereof.

In an eighth embodiment, the present disclosure provides a fluidic device according to any of the first through seventh embodiments, wherein the hydrophilic mask material comprises plasma deposited silicon/oxygen materials or diamond like glass, a nanostructure, a surfactant, a polyester, a polyamide, a polyurethane, a poly(vinyl alcohol), a poly(alkylene glycol), a poly(alkylene oxide), a poly(vinyl pyrrolidone), a rubber elastomer, or a combination thereof.

In a ninth embodiment, the present disclosure provides a fluidic device according to any of the first through eighth embodiments, wherein the first bondable polymeric layer comprises a low density polyethylene, ethylene vinyl acetate, a polyurethane, copolymers of a polyester and a polyolefin, copolymers of a polyurethane and an aromatic poly(meth)acrylate, copolymers of a polycaprolactone and a polyurethane, or a combination thereof.

In a tenth embodiment, the present disclosure provides a fluidic device according to any of the first through ninth embodiments, wherein the second polymeric layer comprises a polyolefin, a polyester, a polyamide, a poly(vinyl chloride), a polyether ester, a polyimide, a polyesteramide, a polyacrylate, a polyvinylacetate, an ethylene acrylic acid adhesive, or a hydrolyzed derivative of polyvinylacetate.

In an eleventh embodiment, the present disclosure provides a fluidic device according to the tenth embodiment, wherein the first bondable polymeric layer has a Vicat softening temperature (T_{g}) of 100 degrees Celsius (C) or less.

In a twelfth embodiment, the present disclosure provides a fluidic device according to any of the first through eighth embodiments, wherein the second polymeric layer comprises a surface treatment comprising a layer of gold.

In a thirteenth embodiment, the present disclosure provides a fluidic device according to any of the first through twelfth embodiments, wherein at least one of the first bondable polymeric layer or the second polymeric layer comprises a reagent configured to react with a sample and provide a response selected from electrochemical, optical, fluorescent, chemiluminescent, or a combination thereof.

In a fourteenth embodiment, the present disclosure provides a fluidic device according to the thirteenth embodiment, wherein at least one of the first bondable polymeric layer or the second polymeric layer further comprises a detector.

In a fifteenth embodiment, the present disclosure provides a fluidic device according to the fourteenth embodiment, wherein the detector comprises an electrode disposed on the first major surface of the first polymer layer or of the second polymeric layer, and wherein the open volume is in fluid communication with the electrode.

In a sixteenth embodiment, the present disclosure provides a fluidic device according to any of the first through fourteenth embodiments, wherein the interstitial space comprises a volume of 500 nanoliters or less per square centimeter of the first portion of the first major surface of the first bondable polymeric layer.

In a seventeenth embodiment, the present disclosure provides a fluidic device according to any of the first through sixteenth embodiments, wherein the first bondable polymeric layer, the second polymeric layer, or both further comprises a fiducial structure or mark.

In an eighteenth embodiment, the present disclosure provides a fluidic device according to any of the first through seventeenth embodiments, further comprising a channel connecting the first portion of the first major surface of the first bondable polymeric layer at least one of a second major surface of the first bondable polymeric layer or a second major surface of the second polymeric layer.

In a nineteenth embodiment, the present disclosure provides a fluidic device according to any of the first through eighteenth embodiments, wherein at least one of the first portion of the first major surface of the first bondable polymeric layer or the second portion of the first major surface of the second polymeric layer exhibits an average surface roughness (Rₐ) of 1 nanometer (nm) to 5 micrometers (μm).

In a twentieth embodiment, the present disclosure provides a fluidic device according to the nineteenth embodiment, wherein the at least one point of contact between the hydrophilic mask material and the second portion of the first major surface occurs at a location of a maximum height of the surface roughness.

In a twenty-first embodiment, the present disclosure provides a fluidic device according to any of the first through twentieth embodiments, comprising a gap between a first edge of the first bondable polymeric layer and a directly adjacent first edge of the second polymeric layer of up to 5 μm.

In a twenty-second embodiment, the present disclosure provides a fluidic device according to any of the first through twenty-first embodiments, wherein the hydrophilic mask material has an average thickness of 1 nm to 1 μm.

### EXAMPLES

Objects and advantages of this disclosure are further illustrated by the following examples, but the particular materials and amounts thereof recited in these examples, as well as other conditions and details, should not be construed to unduly limit this disclosure. Unless otherwise noted or otherwise apparent from the context, all parts, percentages, ratios, etc. in the Examples and the rest of the specification are by weight.

### General Procedure for the Preparation of Fluidic Devices

Fluidic Devices of the examples were prepared with the bondable polymeric layer component of the devices being a two-layer polymeric film that had a 250 micrometer thick ZEONOR 1420R cyclic olefin polymer (Zeon Chemicals L.P., Louisville, KY) backing layer and a 25 micrometer bondable layer of PRIMACOR 3330 polyethylene/acrylic acid copolymer (SK Global Chemical, Seoul, South Korea). The bondable polymeric layer component was prepared by a bi-layer extrusion process. Two single screw extruders were used to feed a 25 cm multi-manifold die. The extrusion process was done horizontally into a nip between two rollers. Extruder conditions are listed in Table 1.

TABLE 1

| | Backing Layer | Bondable Layer |
|---|---|---|
| Zone 1 Temperature | 475° F. (246.1° C.) | 325° F. (162.8° C.) |
| Zone 2 Temperature | 500° F. (260° C.) | 375° F. (190.6° C.) |
| Zone 3 Temperature | 500° F. (260° C.) | 400° F. (204.4° C.) |
| Zone 4 Temperature | 500° F. (260° C.) | 425° F. (218.3° C.) |
| Speed | 11.5 rpm | 16 rpm |
| Neck Tube Temperature | 535° F. (279.4° C.) | 425° F. (218.3° C.) |
| Melt Pressure | 520 psi | 930 psi |

The polymeric layer component used in the devices of the examples was a 75 micrometer thick Melinex 454 polyester PET film (Tekra, New Berlin, WI) that was coated with a thin layer (about 20 nanometers) of gold using vacuum deposition. Gold was applied to one surface of the PET film by placing the film (5.1 cm×5.1 cm section) in a vacuum sputtering chamber (Desk V deposition coater, Denton Vacuum, Moorestown, NJ) for 2 minutes with a power setting of 30 milliamps.

A hydrophilic mask coating bearing silanol and siloxane groups was applied to the bondable layer surface of the bondable polymeric layer component using a parallel plate capacitively coupled plasma reactor as described in U.S. Pat. No. 6,696,157. Prior to placing in the reactor, a patterned mask template was applied over the bondable surface so that the hydrophilic coating could be applied in a specified pattern. The mask template material was 3M 851-ST polyester/silicone tape (3M Company, St. Paul, MN) and each pattern was cut from the tape using a Muse laser cutter (Full Spectrum Laser Company, Las Vegas, NV). Individual patterns used in the examples are shown in FIG. 4A. The patterned mask template was applied to the bondable surface using a hand roller.

The chamber of the reactor had a central cylindrical powered electrode with a surface area of 1.7 m². After placing the film on the powered electrode, the reactor chamber was pumped down to a base pressure of less than 1.3 Pa (2 mTorr). Oxygen and hexamethyldisiloxane (HMDSO) gasses were flowed into the chamber at rates of 200 SCCM (standard cubic centimeters per minute), and 1000 SCCM respectively. Treatment was carried out using a plasma enhanced CVD method by coupling RF power into the reactor at a frequency of 13.56 MHz and an applied power of 7000 watts. Treatment time was controlled by moving the film through the reaction zone at rate of 30 feet/minute (914 cm/minute), resulting in an approximate exposure time of 10 seconds. After the first treatment, the gasses and power were turned off to the reactor chamber. The chamber was then pumped down to a base pressure of 1.3 Pa (2 mTorr). Subsequently, oxygen gas was flowed into the chamber at a rate of 1000 SCCM. A second treatment was carried out using a plasma by coupling RF power into the reactor at a frequency of 13.56 MHz and an applied power of 5000 watts. Treatment time was controlled by moving the film through the reaction zone at rate of 30 feet/minute (914 cm/minute), resulting in an approximate exposure time of 10 seconds. At the end of this treatment time, the RF power and the gas supply were stopped and the chamber was returned to atmospheric pressure. The patterned mask template was then removed to provide the hydrophilic mask coating on the bondable layer surface in a shape defined by the patterned mask template. The thickness of the deposited hydrophilic mask coating was about 20-50 nm.

The bondable polymeric layer component and the polymeric layer component were laminated together so that the surface of the bondable layer with the hydrophilic mask coating faced the gold coated surface of the PET film. To laminate, the film stack was placed on a heated surface (110° C.) with the PET polymer layer in contact with the heated surface. After 30 seconds on the heated surface, a weighted roller (2.0 kg) was passed over the film stack 3-times with a back and forth motion. The resulting laminated sample was removed from the heated surface and allowed to cool to room temperature. Finished devices were cut from the laminated samples.

### Example 1

The laminated sample 400b shown in FIG. 4B was prepared using the general procedure described above. A fluidic device 400c (FIG. 4C) defined by the overlaid dotted lines 407 of FIG. 4B (external dimensions of 15 mm×8 mm) was cut from the laminated sample 400b using a razor blade. The overlaid dotted lines were added to the photographic image as a visual representation of the section cut from the laminated sample. The hydrophilic mask material area 414 formed an L-shaped channel that had a first channel section of about 11 mm by 2 mm extending from edge 411 with a narrower orthogonal channel section of about 4 mm by 1 mm extending from the first section to the third edge 415.

A droplet of deionized water was contacted with the hydrophilic material edge opening 411 (FIG. 4C) using a transfer pipette (2 mL graduated, Molecular Bio Products, Inc., San Diego CA). Water was aspirated into the pipette, followed by application of pressure to the pipette bulb to generate an approximately 10 microliter hanging droplet at the pipette tip. Following contact of the hanging droplet with the edge opening, the advancing meniscus of the water was monitored using a Zeiss Lumar V12 stereo microscope (Zeiss, Dublin, CA) (12× magnification) equipped with an Axiocam HSM camera (Zeiss) and operated at a 30 millisecond frame rate. Images were processed using Axiovision software (Zeiss). Images of fluid movement in the device are shown in FIGS. 4C-4E.

FIG. 4B is a photograph of the sample 400b of Example 1 prior to cutting the exemplary fluidic device from the remainder of the sample. More particularly, in the photograph of the sample 400b various features are visible, including the hydrophilic mask material 414 having a shape of a channel with a 90 degree turn 409. The thick dotted line 407 indicates the perimeter to be cut to form a fluidic device from the sample 400b. Once cut, a fluid sample can be wicked into the fluidic device by contacting the fluid with either the first edge 411 or the third edge 415 in the location of the hydrophilic mask material 414. Whichever edge is used, the design of the hydrophilic mask material 414 allows for gas and fluid to vent out the other edge. The gold material has a darker shade than the hydrophilic mask material 414 and indicates the bonded portions 418 of the sample 400b surrounding the hydrophilic mask material 414.

FIG. 4C is a stereo microscope image of a portion of the exemplary fluidic device 400c of Example 1 prior to contact with deionized water. The hydrophilic mask material 414 can be seen to be surrounded by the bonded portion 418 except at the first edge 411 and the third edge 415 of the fluidic device 400c. FIG. 4D is a stereo microscope image of a portion of the fluidic device of Example 1 after 1.685 seconds of contact with the water. In addition to the hydrophilic mask material 414 and the bonded portion 418, a volume of water 495 is visible on the hydrophilic mask material 414, having wicked a distance along the open volume provided by the interstitial space between the hydrophilic mask material 414 and a second polymeric layer (not visible), from the first edge 411 to the location of the large arrow 421d partway along the linear channel. FIG. 4E is a stereo microscope image of a portion of the fluidic device of Example 1 after 3.370 seconds of contact with the water. In the fluidic device 400e, it can be seen that additional wicking time has resulted in the water 495 traveling further in the open volume, from the first edge 411, along the channel, and past the 90 degree turn, towards the third edge 415, at the location of the large arrow 421e.

### Example 2

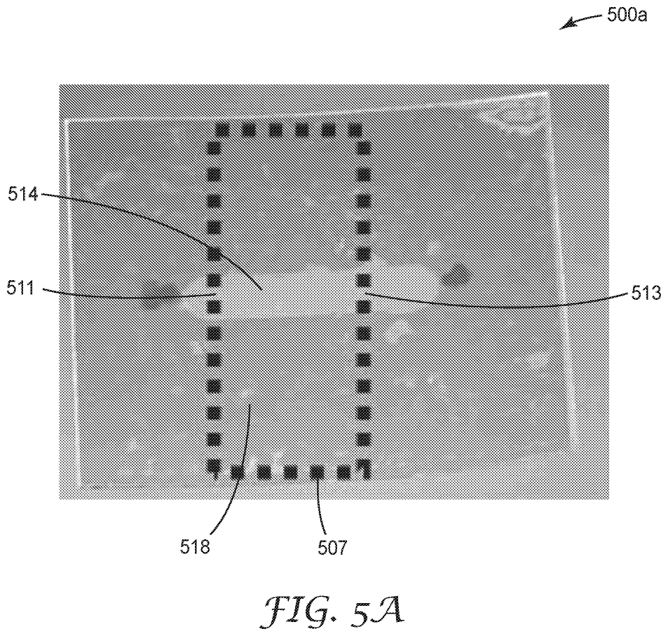

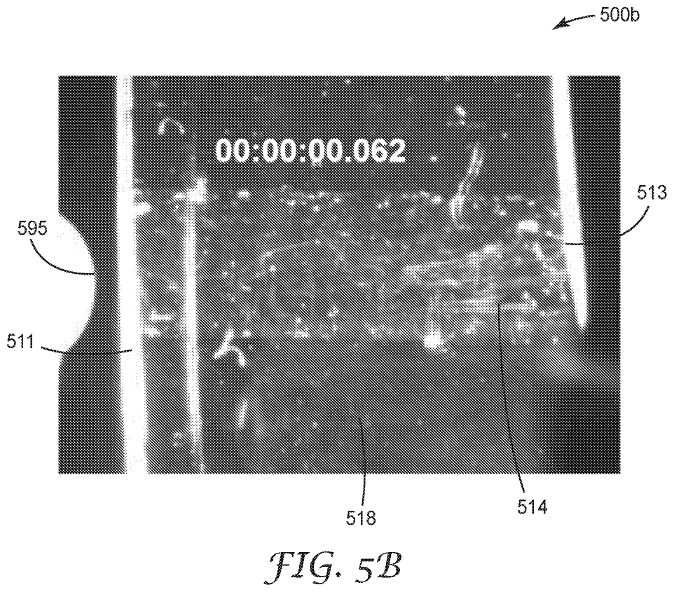

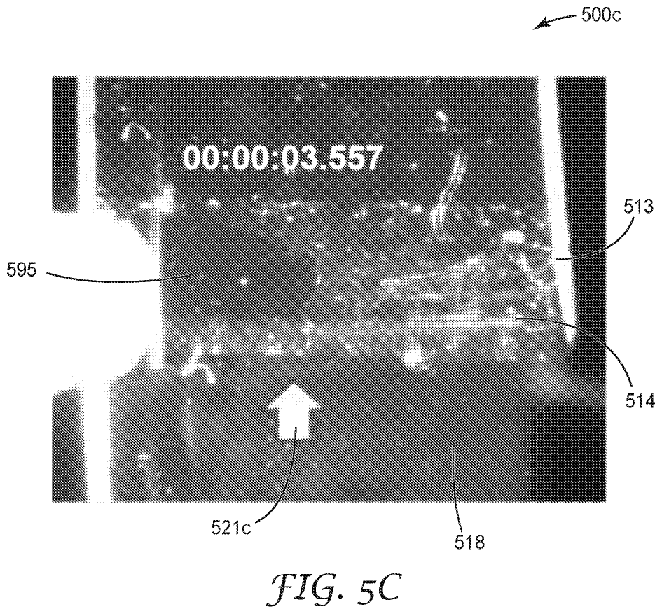

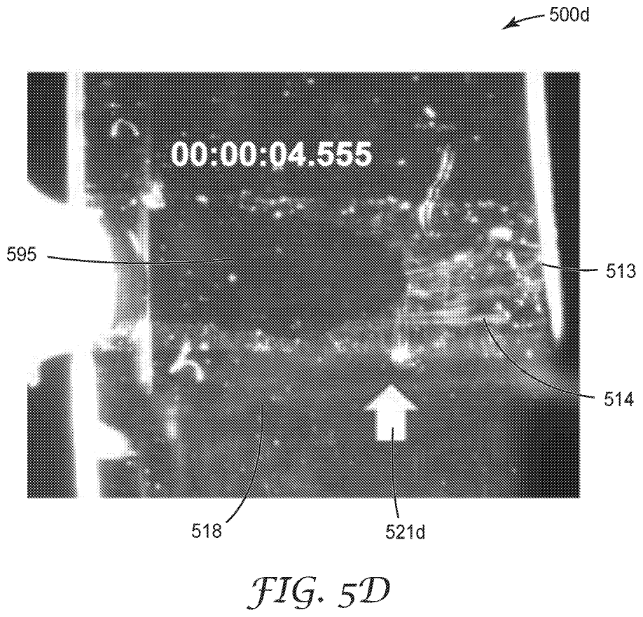

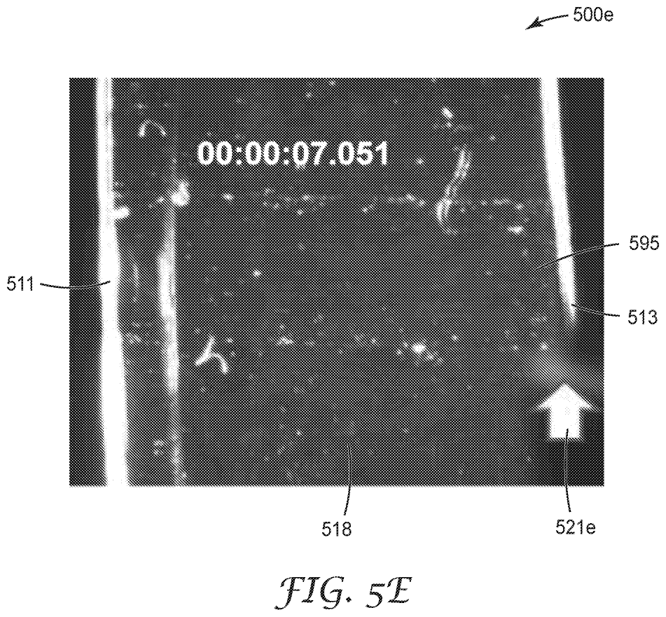

The laminated sample 500a shown in FIG. 5A was prepared using the general procedure described above. A fluidic device 500b (FIG. 5B) with a perimeter defined by the overlaid dotted lines 507 of FIG. 5A (external dimensions of 15 mm×8 mm) was cut from the laminated sample 500a using a razor blade. The hydrophilic mask material 514 formed a straight channel that was 2 mm wide and extended from the first edge 511 to the second edge 513.

A droplet of deionized water was contacted with the hydrophilic material edge opening 511 (FIG. 5A) according to the method described in Example 1. Following contact of the hanging droplet with the edge opening, the advancing meniscus of the water was monitored according to the method described in Example 1. Images of fluid movement in the device are shown in FIGS. 5B-5E.

FIG. 5A is a photograph of the sample of Example 2 prior to cutting the exemplary fluidic device from the remainder of the sample. More particularly, in the photograph of the sample 500a various features are visible, including the hydrophilic mask material 514 having a shape of a straight channel. The thick dotted line 507 indicates the perimeter to be cut to form a fluidic device from the sample 500a. Once cut, a fluid sample can be wicked into the fluidic device by contacting the fluid with either the first edge 511 or the second edge 513 in the location of the hydrophilic mask material 514. Whichever edge is used, the design of the hydrophilic mask material 514 allows for gas and fluid to vent out the other edge. The darker shade of the gold material than the hydrophilic mask material 514 indicates the bonded portions 518 of the sample 500a on either side of the hydrophilic mask material 514.

FIG. 5B is a stereo microscope image of a portion of the fluidic device 500b of Example 2 after 0.062 seconds of contact with deionized water. The hydrophilic mask material 514 can be seen to have the shape of a channel with the bonded portion 518 on either side of the hydrophilic mask material 514. A volume of the water 595 is visible starting to wick into the first edge 511 of the fluidic device 500b at the beginning of the hydrophilic mask material 514. FIG. 5C is a stereo microscope image of a portion of the fluidic device 500c of Example 2 after 3.577 seconds of contact with the water. By this time, the water 595 had wicked a distance along the open volume provided by the interstitial space between the hydrophilic mask material 514 and a second polymeric layer (not visible), from the first edge 511 to the location of the large arrow 521c partway along the linear channel towards the second edge 513. FIG. 5D is a stereo microscope image of a portion of the fluidic device of Example 2 after 4.555 seconds of contact with the water. The fluid 595 had wicked a further distance along the open volume, at the location of the large arrow 521d closer towards the second edge 513. FIG. 5E is a stereo microscope image of a portion of the fluidic device 500e of Example 2 after 7.051 seconds of contact with the water. By this time, the water 595 had wicked all the way to the second edge 513, at the location of the large arrow 521c.

### Example 3

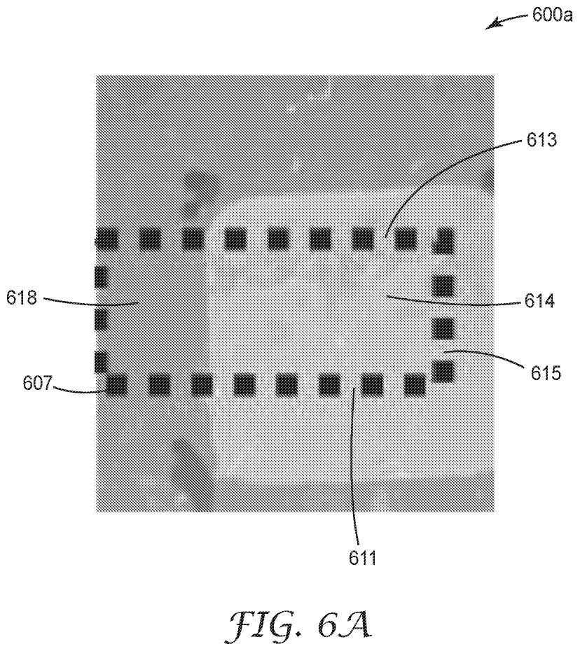

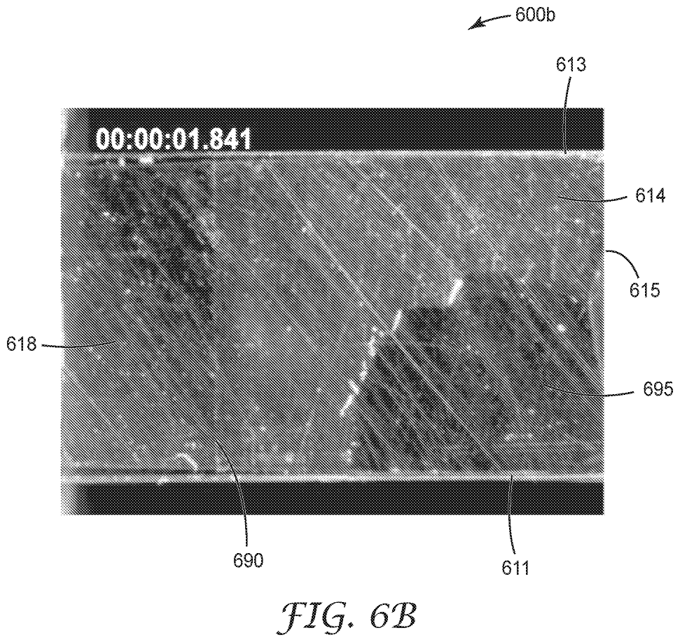

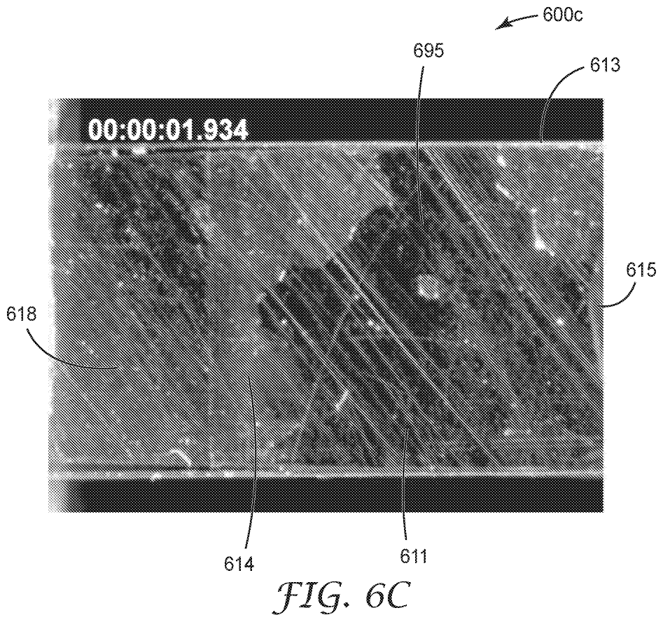

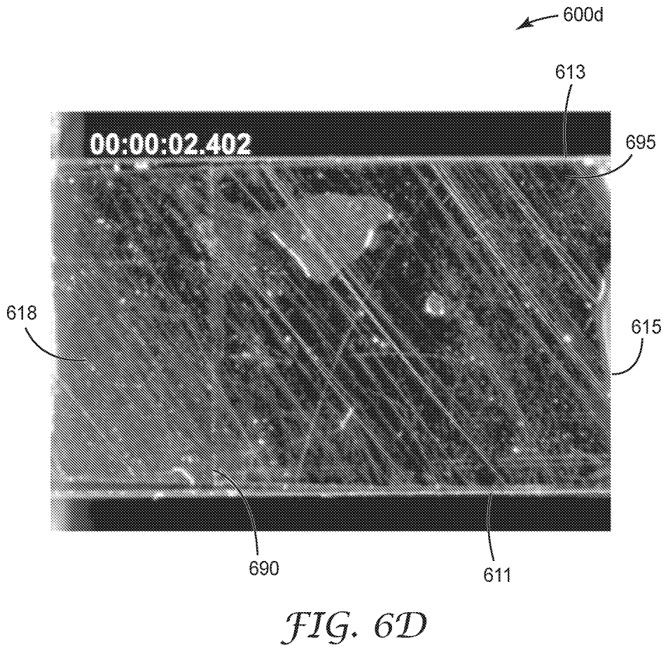

The laminated sample 600a shown in FIG. 6A was prepared using the general procedure described above. A fluidic device 600b (FIG. 6B) with a perimeter defined by the overlaid dotted lines 607 of FIG. 6A (external dimensions of 15 mm×8 mm) was cut from the laminated sample 600a using a razor blade. The hydrophilic mask material 614 formed a rectangular channel of 8 mm by 8 mm that was edge aligned with the first, second, and third edges (611, 613, 615).

A droplet of deionized water was contacted with the hydrophilic material edge opening 615 (FIG. 6B) according to the method described in Example 1. Following contact of the hanging droplet with the edge opening, the advancing meniscus of the water was monitored according to the method described in Example 1. Images of fluid movement in the device are shown in FIGS. 6B-6D.

FIG. 6A is a photograph of the sample of Example 3 prior to cutting the exemplary fluidic device from the remainder of the sample. More particularly, in the photograph of the sample 600a various features are visible, including the hydrophilic mask material 614 having a shape of a rectangle. The thick dotted line 607 indicates the perimeter to be cut to form a fluidic device from the sample 600a. Once cut, a fluid sample can be wicked into the fluidic device by contacting the fluid with any of the first edge 611, the second edge 613, or the third edge 615 in the location of the hydrophilic mask material 614. The hydrophilic mask material 614 has a continuous shape that extends fully along the first edge 611, along the opposing second edge 613, and along the third edge 615 located between the first edge 611 and the second edge 613. This configuration allows for venting of air and/or liquid at any point along each of the edges during use of the fluidic device. The darker shade of the gold material than the hydrophilic mask material 614 indicates the bonded portion 618 of the sample 600a adjacent to the hydrophilic mask material 614.

FIG. 6B is a stereo microscope image of a portion of the fluidic device 600b of Example 3 after 1.841 seconds of contact with deionized water. The hydrophilic mask material 614 can be seen to have the shape of a rectangle with the bonded portion 618 to the left of the hydrophilic mask material 614 and an interface 690 between the hydrophilic mask material 614 and the bonded portion 618. A volume of the water 695 is visible starting to wick into the third edge 615 of the fluidic device 600b along the open volume provided by the interstitial space between the hydrophilic mask material 614 and a second polymeric layer (not visible). FIG. 6C is a stereo microscope image of a portion of the fluidic device 600c of Example 3 after 1.934 seconds of contact with the water. The water 695 had wicked a further distance along the open volume towards the interface 690 and reaching the second edge 613 opposite the first edge 611. FIG. 6D is a stereo microscope image of a portion of the fluidic device 600e of Example 3 after 2.402 seconds of contact with the water. By this time, some of the water 695 had wicked all the way to the interface 690 between the hydrophilic mask material and the bonded portion 618.

### Example 4

A melt-bondable film was prepared using compression molding. The upper and lower press platens of a hydraulic press were heated to 230° F. (110° C.). A sheet of polypropylene (C700-35N, Braskem, Philadelphia PA) was placed on the lower platen. Pellets of PEARLBOND 1160L polyurethane melt polymer (Lubrizol, Wickliffe, OH) were placed on the polypropylene sheet to form a dense packed monolayer with a diameter of approximately 10 cm. A sheet of MELINEX 454 polyester PET (Tekra, 5 mil thick) was placed over the pellets and a stainless steel sheet (0.8 mm thick) was placed over the PET sheet. The platens were closed to a pressure of 10,000 pounds for 5 minutes followed by cooling to 70° F. (21.1° C.) under pressure. After cooling the platens were separated and the bondable film was peeled from the polypropylene layer. A hydrophilic mask coating bearing silanol and siloxane groups was applied to the bondable film according to the procedure described in the section ‘General Procedure for the Preparation of Fluidic Devices’ using the patterned mask 600 described in FIG. 4A.

The lower platen of a press was heated to 50° C. and a section of the bondable film was placed on the heated lower platen for 30 seconds with the hydrophilic material treated surface of the film facing away from the lower platen surface. A 5 mil thick sheet of MELINEX 454 polyester PET film (Tekra) was placed over the bondable film. The press was closed and held using hand pressure to laminate the films. The resulting laminated sample was removed from the press and allowed to cool to room temperature. The laminated sample was cut with a razor blade to provide the fluid device 200 described in FIG. 2A. A 3 microliter droplet of citrated human blood was contacted with the hydrophilic edge opening 215 (FIG. 2A) of the fluid device using a transfer pipette. Following contact of the blood sample with the edge opening, the advancing meniscus of blood was monitored according to the method described in Example 1. Images of fluid movement in the device are shown in FIGS. 2B-2C.

All of the patents and patent applications mentioned above are hereby expressly incorporated by reference. The embodiments described above are illustrative of the present invention and other constructions are also possible. Accordingly, the present invention should not be deemed limited to the embodiments described in detail above and shown in the accompanying drawings, but instead only by a fair scope of the claims that follow along with their equivalents.

1. A fluidic device comprising:
a) a first bondable polymeric layer having a first major surface that is substantially planar;
b) a second polymeric layer having a first major surface that is substantially planar; and
c) a hydrophilic mask material disposed on a first portion of the first major surface of the first bondable polymeric layer, wherein a surface of the hydrophilic mask material exhibits an advancing contact angle with water of less than 90 degrees;
wherein a second portion of the first major surface of the first bondable polymeric layer is bonded to a first portion of the first major surface of the second polymeric layer, wherein the hydrophilic mask material and a second portion of the first major surface of the second polymeric layer are in direct contact with each other at at least one point, wherein an open volume is defined by interstitial space located between the hydrophilic mask material and the second portion of the first major surface of the second polymeric layer, wherein the open volume comprises two or more openings and wherein at least one of the openings is located at an edge of the first bondable polymeric layer.

2. The fluidic device of claim 1, wherein the first portion of the first major surface of the first bondable polymeric layer on which the hydrophilic mask material is disposed is a continuous area that extends from a first edge of the first bondable polymeric layer to each of an opposing second edge of the first bondable polymeric layer and a third edge of the first bondable polymeric layer that is located between the first edge and the second edge of the first bondable polymeric layer.

3. The fluidic device of claim 2, wherein the first portion extends along an entirety of the third edge.

4. The fluidic device of claim 1, wherein the first portion of the first major surface of the first bondable polymeric layer on which the hydrophilic mask material is disposed is a pattern that defines a fluid path from:
a) a first portion of the third edge of the first bondable polymeric layer to i) at least one of the first edge or the second edge of the first bondable polymeric layer or ii) back to the third edge of the first bondable polymeric layer at a location spaced apart from the first portion of the third edge;
b) a first portion of the second edge of the first bondable polymeric layer to i) at least one of the first edge or the third edge of the first bondable polymeric layer or ii) back to the second edge of the first bondable polymeric layer at a location spaced apart from the first portion of the second edge; or
c) a first portion of the first edge of the first bondable polymeric layer to i) at least one of the second edge or the third edge of the first bondable polymeric layer or ii) back to the first edge of the first bondable polymeric layer at a location spaced apart from the first portion of the first edge.

5. The fluidic device of claim 4, wherein the first portion of the first major surface of the first bondable polymeric layer has a shape that has a length that is at least 10 times greater than a width of the shape.

6. The fluidic device of claim 4, wherein the first portion of the first major surface of the first bondable polymeric layer has a linear shape that comprises a straight portion, a curved portion, or combinations thereof.

7. The fluidic device of claim 1, wherein the hydrophilic mask material comprises plasma deposited silicon/oxygen materials or diamond like glass, a nanostructure, a surfactant, a polyester, a polyamide, a polyurethane, a poly(vinyl alcohol), a poly(alkylene glycol), a poly(alkylene oxide), a poly(vinyl pyrrolidone), a rubber elastomer, or a combination thereof.

8. The fluidic device of claim 1, wherein the first bondable polymeric layer comprises a low density polyethylene, ethylene vinyl acetate, ethylene acrylic acid, a polyurethane, copolymers of a polyester and a polyolefin, copolymers of a polyurethane and an aromatic poly(meth)acrylate, copolymers of a polycaprolactone and a polyurethane, or a combination thereof.

9. The fluidic device of claim 1, wherein the second polymeric layer comprises a polyolefin, a polyester, a polyamide, a poly(vinyl chloride), a polyether ester, a polyimide, a polyesteramide, a polyacrylate, a polyvinylacetate, an ethylene acrylic acid adhesive, or a hydrolyzed derivative of polyvinylacetate or a combination thereof.

10. The fluidic device of claim 9, wherein the first bondable polymeric layer has a Vicat softening temperature (T_{g}) of 100 degrees Celsius (C) or less.

11. The fluidic device of claim 1, wherein at least one of the first bondable polymeric layer or the second polymeric layer comprises a reagent configured to react with a sample and provide a response selected from electrochemical, optical, fluorescent, chemiluminescent, or a combination thereof.

12. The fluidic device of claim 11, wherein at least one of the first bondable polymeric layer or the second polymeric layer further comprises a detector.

13. The fluidic device of claim 1, wherein the interstitial space comprises a volume of 500 nanoliters or less per square centimeter of the first portion of the first major surface of the first bondable polymeric layer.

14. The fluidic device of claim 1, further comprising a channel connecting the first portion of the first major surface of the first bondable polymeric layer at least one of a second major surface of the first bondable polymeric layer or a second major surface of the second polymeric layer.

15. The fluidic device of claim 1, wherein at least one of the first portion of the first major surface of the first bondable polymeric layer or the second portion of the first major surface of the second polymeric layer exhibits an average surface roughness (Rₐ) of 1 nanometer (nm) to 5 micrometers (μm) and wherein the at least one point of contact between the hydrophilic mask material and the second portion of the first major surface occurs at a location of a maximum height of the surface roughness.

16. The fluidic device of claim 1, comprising a gap between a first edge of the first bondable polymeric layer and a directly adjacent first edge of the second polymeric layer of up to 5 μm.

17. The fluidic device of claim 1, wherein the hydrophilic mask material has an average thickness of 1 nm to 1 μm.
